(19) United States
(12) Patent Application Publication
(10) Pub. No.: US 2022/0287644 A1
(43) Pub. Date: Sep. 15, 2022

(54) MOVEMENT COMPENSATION FOR VOLTAGE MEASURING SYSTEMS

(71) Applicant: Siemens Healthcare GmbH, Erlangen (DE)

(72) Inventors: Ulrich BATZER, Spardorf (DE); Peter GREIF, Pinzberg/Gosberg (DE)

(73) Assignee: Siemens Healthcare GmbH, Erlangen (DE)

(21) Appl. No.: 17/687,749

(22) Filed: Mar. 7, 2022

(30) Foreign Application Priority Data
Mar. 10, 2021 (DE) 10 2021 202 347.9

(51) Int. Cl. A61B 5/00; A61B 5/263; A61B 5/30

(52) U.S. Cl. CPC A61B 5/721; A61B 5/263; A61B 5/30

(57) ABSTRACT

At least one example embodiment relates to a measuring system for measuring bioelectrical signals of a patient, the measuring system comprising a sensor electrode, and a mechanical mounting for the sensor electrode, the mechanical mounting being at least partially compressible and comprising a frame structure and a supporting structure. The mechanical mounting is fastened to a substrate of the measuring system and supports the sensor electrode against the substrate, the supporting structure is arranged beneath the sensor electrode, the frame structure at least partially surrounds the supporting structure, and the supporting structure is configured higher than the frame structure.

### PRIORITY STATEMENT

The present application hereby claims priority under 35 U.S.C. § 119 to German patent application number DE 102021202347.9 filed Mar. 10, 2021, the entire contents of which are hereby incorporated herein by reference.

### FIELD

Example embodiments generally relate to measuring systems for measuring bioelectrical signals from a patient, which compensates for movement-related disturbance effects, corresponding signal measuring circuits and corresponding differential voltage measuring systems.

### BACKGROUND

Voltage measuring systems, in particular differential voltage measuring systems for measuring bioelectrical signals, are used, for example, in the medical field for measuring electrocardiograms (ECG), electroencephalograms (EEG) or electromyograms (EMG).

The measurement of a heart activity with the a voltage measuring system is may be used for imaging of the heart in order to adapt the imaging process to the very pronounced movement of the heart during the heartbeat. For this, conventional sensors which must be fastened to the body of the patient are used. One possibility for heartbeat measurement is a capacitive ECG in which an ECG signal is detected purely capacitively without directly contacting the patient with the sensor, in particular through the clothing of the patient. In order to achieve a good signal quality of the heartbeat signal, the signal amplitude must preferably be large. This can be achieved with a high capacitance between the patient and the sensor. The capacitance can be influenced by the size of the coupling area between the sensor and the patient. The larger the coupling area is selected to be, the greater also is the capacitance achieved.

For the patient it is particularly comfortable if the capacitive ECG measurement can take place without the placement or fastening of individual sensors. For this purpose, it is known to integrate the sensor equipment into the surface of a patient couch of, for example, an imaging system or into an underlay mat or a seat backrest so that the voltage measurement can take place as soon as the patient is positioned on the patient couch or the seat. An apparatus of this type is described, for example, in the German patent application DE 10 2015 218 298 B3.

Alternatively, ECG chest belts are known which are stretched round the ribcage of the patient and typically have electrical contacts/electrodes at two lateral positions on the ribcage for capacitive ECG signal measurement.

Both variants have the problem that disturbances of the measurement signals can occur since, due to movements, a variable contact pressure prevails between the patient and the capacitive sensors. In this way, the complex impedance between the patient and the sensor changes. Triboelectric effects can occur in the clothing and possible other layers between the sensor and the patient and within the sensor itself. These significantly impair the signal quality.

In order to compensate for already existing electrical charges and/or tribologically-caused disturbances, for example, a solution according to the German utility model DE 20 2020 101579 U1 has been proposed.

With ECG chest belts, provision has also been made to attach them very firmly round the ribcage. Chest belts are typically configured to be stretchable, in order, for example, partially to absorb a movement caused by a breathing-related extension of the ribcage.

### SUMMARY

Nevertheless, the contact pressure on the sensors is thereby changed.

By contrast thereto, at least one example embodiment permits a relatively high signal quality to be achieved in the derivation of bioelectrical signals by differential voltage measuring systems. In particular, at least one example embodiment reduces and/or minimizes movement-related pressure changes on a capacitive sensor in advance and reduces/prevents disturbing influences on bioelectrical signals.

Example embodiments provide a measuring system for measuring bioelectric measurement signals from a patient, a signal measuring circuit for a differential voltage measuring system for measuring bioelectrical measurement signals from a patient and a differential voltage measuring system.

Features, advantages or alternative embodiments which can be directed, for example, to a method are also described or claimed in conjunction with one of the apparatuses. The corresponding functional features are thereby provided by way of corresponding physical modules or units of the apparatuses.

At least one example embodiment provides a measuring system for measuring bioelectrical signals of a patient, the measuring system including a sensor electrode and a mechanical mounting for the sensor electrode, the mechanical mounting being at least partially compressible and comprising a frame structure and a supporting structure, wherein the mechanical mounting is fastened to a substrate of the measuring system and supports the sensor electrode against the substrate, the supporting structure is arranged beneath the sensor electrode, the frame structure at least partially surrounds the supporting structure, and the supporting structure is configured higher than the frame structure.

In an example embodiment, the mechanical mounting is formed at least partially of foam material.

In an example embodiment, the supporting structure has a lower hardness than the frame structure.

In an example embodiment, the mechanical mounting comprises a carrier structure which extends beneath the supporting structure and the frame structure.

In an example embodiment, the mechanical mounting comprises an intermediate structure which connects the carrier structure to the frame structure and the supporting structure.

In an example embodiment, the intermediate structure is configured integrally with the frame structure.

In an example embodiment, the mechanical mounting comprises a comfort structure arranged above the frame structure, wherein the supporting structure is higher than the frame structure and the comfort structure together.

In an example embodiment, the supporting structure has a lower hardness than at least one of the comfort structure or the carrier structure.

In an example embodiment, the frame structure, or the frame structure and the comfort structure, have a recess facing in the direction of the sensor electrode, in which the supporting structure can give way depending upon a compression.

In an example embodiment, the supporting structure is formed from a foam material, the foam material configured to create a substantially constant counterforce based on a compression change.

In an example embodiment, the height of the supporting structure is approximately 1.4 to 1.6 times the height of the frame structure or the frame structure and the comfort structure together.

In an example embodiment, the system further includes a compression film, the compression film being arrange to precompress the supporting structure by approximately 30% to 40%.

At least one example embodiment provides a signal measuring circuit for a differential voltage measuring system for measuring bioelectrical signals of a patient, the signal measuring circuit comprising a measuring system, a measuring amplifier circuit and a sensor line between the measuring amplifier circuit and the sensor electrode.

At least one example embodiment provides a differential voltage measuring system for measuring bioelectrical signals of a patient, which has at least two signal measuring circuits, each signal measuring circuit corresponding to one useful signal path, wherein at least one of the signal measuring circuits comprises the measuring system.

At least one example embodiment provides a differential voltage measuring system, comprising at least two signal measuring circuits, each of the measuring circuits including, a sensor electrode, and a mechanical mounting for the sensor electrode, the mechanical mounting being at least partially compressible and comprising a frame structure and a supporting structure, the mechanical mounting is fastened to a substrate of the measuring system and supports the sensor electrode against the substrate, the supporting structure is arranged beneath the sensor electrode, the frame structure at least partially surrounds the supporting structure, and the supporting structure is configured higher than the frame structure; and the frame structures of the at least two measuring systems are each configured integrally with one another.

In an example embodiment, the supporting structure has a lower hardness than the frame structure.

In an example embodiment, the supporting structure is formed from a foam material, the foam material configured to create a substantially constant counterforce based on a compression change.

### BRIEF DESCRIPTION OF THE DRAWINGS

The above-described properties, features and advantages of example embodiments and the manner in which they are achieved are made more clearly and distinctly intelligible with the following description of the exemplary embodiments which are described in greater detail making reference to the drawings. This description entails no limitation of example embodiments. In different figures, the same components are provided with identical reference signs. The drawings are, in general, not to scale. In the drawings:

FIG. 1 is a view of a measuring system in an exemplary embodiment,

FIG. 2 is a view of a measuring system in a further exemplary embodiment,

FIG. 3 is a view of a measuring system in a further exemplary embodiment relating to different loading states,

FIG. 4 is an exemplary compressive strength curve for a supporting structure material of a measuring system in an exemplary embodiment,

FIG. 5 is a view of a differential voltage measuring system in a first exemplary embodiment,

FIG. 6 is a view of a differential voltage measuring system comprising two signal measuring circuits according to the invention in another exemplary embodiment,

FIG. 7 is a comparison of differently acquired ECG signals,

FIG. 8 is a view of a measuring system in another exemplary embodiment, and

FIG. 9 is a view of a differential voltage measuring system comprising two signal measuring circuits according to the invention in another exemplary embodiment.

### DETAILED DESCRIPTION

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one example embodiment provides a measuring system for measuring bioelectrical measurement signals from a patient.

The patient is typically a person. In principle, the patient can also be an animal.

The measuring system comprises a sensor electrode. The measuring system also comprises a mechanical, at least partially compressible mounting for the sensor electrode comprising a frame structure and a supporting structure. The mechanical mounting is therein fastened to a substrate of the measuring system and is constructed such that it supports the sensor electrode against the substrate. The supporting structure is arranged beneath the sensor electrode and the frame structure at least partially surrounds the supporting structure. The supporting structure is constructed higher as compared with the frame structure.

In at least one example embodiment, the sensor electrode is configured as part of a signal measuring circuit of a differential voltage measuring system.

Accordingly, at least one example embodiment relates to a signal measuring circuit for a differential voltage measuring system for measuring bioelectrical signals from a patient. The signal measuring circuit comprises:
a measuring system according to the invention,
a measuring amplifier circuit, and
a sensor line between the measuring amplifier circuit and the sensor electrode.

In some example embodiments, the sensor electrode is configured as a flat electrode, preferably as a square or rectangular or round flat electrode and has a film-like structure.

The sensor line serves in some example embodiments for transferring the bioelectrical measurement signals acquired by the sensor electrode to the measuring amplifier circuit.

The measuring amplifier circuit preferably comprises an operational amplifier which can be configured as a so-called follower. This means that the negative input of the operational amplifier, also known as the inverting input, is coupled to the output of the operational amplifier, whereby a high virtual input impedance is generated at the positive input.

The mechanical mounting of the sensor electrode according to at least one example embodiment is configured at least partially compressible, i.e. on mechanical loading caused, in particular, by a patient leaning against the measuring system or positioned thereon, the mechanical mounting advantageously changes its shape, in particular its height, at least in the compressibly configured regions or parts. The mechanical mounting connects a substrate of the measuring system, for example, a lower outer surface of the measuring system to the sensor electrode. The mechanical mounting therefore carries and supports the sensor electrode without pressure/loading at a previously defined spacing from the substrate. For stabilizing the measuring system, the mounting is fastened, for example, welded or glued onto the substrate.

In order to minimize pressure changes on the sensor electrode induced by a patient movement and capacitance changes induced thereby and tribological effects, the mechanical mounting comprises a frame structure and a supporting structure. The supporting structure is arranged beneath the sensor electrode. The supporting structure thus has substantially the same base area as the sensor electrode. If the measuring system is integrated into a mat-like structure or if no size or space details are to be taken into account, the sensor electrode and the supporting structure preferably have a square base area of between 3 cm×3 cm and 7 cm×7 cm. Particularly preferably, the supporting structure and the sensor electrode each have a base area of 4 cm×4 cm. If the measuring system is a constituent part of a signal measuring circuit for a chest belt, the base area is correspondingly more compact. For this purpose, the sensor electrode and the supporting structure preferably have a base area from 1 cm×1 cm to a maximum of 2 cm×5 cm. The sensor electrode preferably lies in a full-surface manner on the supporting structure. The frame structure at least partially surrounds the supporting structure. In some preferred embodiments, the frame structure surrounds the supporting structure at least on two opposite sides. In other preferred embodiments, the frame structure surrounds the supporting structure completely. Here, the frame structure is therefore a structure formed closed around the supporting structure. In some embodiments of the measuring system for a mat-like structure, the frame structure preferably has a width/thickness in the direction perpendicular to the supporting structure of 1 cm. Overall, the dimensions of the base area of a measuring system according can preferably be between 3 cm (in a spatial direction without a frame structure) or between 5 cm and 9 cm. In embodiments of the measuring system for a chest belt, the frame structure preferably has a thickness/width of 5 mm to 15 mm.

The supporting structure is configured higher than the frame structure. In this way, the supporting structure raises the sensor electrode beyond the frame structure.

In some example embodiments, the mechanical mounting is formed at least partially of foam material. In particular, the supporting structure is formed, from a compressible foam material, preferably a viscoelastic foam material or a polyurethane foam material.

In further example embodiments, the supporting structure has a lower hardness than the frame structure. This can be achieved in that the frame structure is formed from a harder foam material, from plastics, from wood or another incompressible material. Preferably, the material of the frame structure is formed incompressible or only slightly compressible. For enhanced patient comfort, in a measuring system for an ECG mat, the frame structure also consists of a slightly compressible foam material. In a measuring system provided for an ECG chest belt, the frame structure preferably consists of a polyethylene plastics material.

If the mechanical mounting and/or the sensor electrode is now loaded, in particular, by being touched by a patient, for example, if he lies or leans on the sensor electrode, firstly the supporting structure is compressed by the weight force or contact pressure to the height of the frame structure. The force component that is necessary therefor is preset by the height difference between the supporting structure and the frame structure and/or the compressive strength of the supporting structure material. The remaining and typically far greater portion of the force applied in total to the measuring system, however, is diverted into the harder frame structure. The supporting structure is not, or only insignificantly, further compressed by the remaining force portion.

The force component that is necessary to compress the supporting structure to the height of the frame structure is now advantageously independent of the overall force acting on the measuring system. The supporting structure creates a counterforce acting against this force component and of corresponding size. In this compression state of the supporting structure, this is advantageously approximately constant. The mechanical mounting thus causes a decoupling between the compressive force placed upon the sensor electrode and the force acting altogether on the measuring system. The force components overcoming the height difference between the supporting structure and the frame structure on one side and the counterforce of the supporting structure on the other side are in equilibrium. They bring about a substantially constant pressing force acting upon the sensor electrode, a reduction in capacitance changes, a minimizing of triboelectric effects in the clothing of the patient and, thereby, a reduction in disturbances of the measurement signal.

In further example embodiments, in particular embodiments provided for an ECG mat, the mechanical mounting comprises a carrier structure which extends beneath the supporting structure and the frame structure. The carrier structure is preferably constructed layered and in particular in a full-surface manner, i.e. it has a base area corresponding at least to the base area of the measuring system. In some embodiments, the base area of the carrier structure can also be configured larger. In some embodiments, the carrier structure connects the substrate of the measuring system to the frame structure and/or the supporting structure. The carrier structure is preferably connected, preferably welded or glued, both to the substrate and also to the frame structure and supporting structure.

In some embodiments, it is provided that the supporting structure has a lower hardness than the carrier structure. The carrier structure is thus preferably also configured to be compressible, and is preferably also formed from a foam material. The carrier structure serves for flexible mounting of the supporting structure and/or the frame structure. Preferably, the carrier structure is formed from a foam material that is 20% to 30% harder than the foam material of the supporting structure. In some embodiments, the carrier structure serves, alongside the often incompressibly constructed frame structure, to absorb the force applied to the measuring system once the supporting structure has already been compressed to the height of the frame structure. Dependent upon the material selected for the carrier structure, the carrier structure can have a height of between 1 cm and 7 cm.

In further embodiments, in particular, embodiments provided for an ECG mat, the mechanical mounting comprises an intermediate structure which is arranged above the carrier structure. In some embodiments, the intermediate structure connects the carrier structure to the frame structure and/or the supporting structure. The intermediate structure can also be welded or glued to the structures extending below or above. The intermediate structure advantageously has the base area of the carrier structure. It is therefore also constructed layered and also full-surface in some embodiments. I.e. it has no recesses. In other embodiments, the intermediate structure can also be restricted to the base area formed by the base areas of the frame structure and the supporting structure. The intermediate structure is preferably configured thinner than the carrier structure and for that reason alone is slightly compressible. It serves for stabilizing the frame structure and the supporting structure and causes, for example, a more even force input into the carrier structure during patient movement. The intermediate structure can also be formed from a foam material. Alternatively, the intermediate structure consists of a textile-like or fleece-like material and in some embodiments it can also consist of a metallic layer. Dependent upon the material selected, the intermediate structure can have a height of 1-10 mm.

In some example embodiments, the intermediate structure is configured integrally with the frame structure. Alternatively, the frame structure can be connected, in particular welded, on its side facing the carrier structure to the intermediate structure. In this embodiment, the intermediate structure consists of the same slightly compressible or incompressible material as the frame structure and the base area of the intermediate structure corresponds to the base area of the frame structure and the supporting structure. In this embodiment, the frame structure and the intermediate structure form a basket-like shape into which the supporting structure can be inserted during assembly. In this way, the connection between the supporting structure and the frame structure can be achieved purely by insertion of the supporting structure and the clamping effect achieved thereby. Gluing or welding of the frame structure to the supporting structure, as in other embodiments, can be omitted in this case.

In a further embodiment, in particular embodiments that are provided for an ECG mat, the mechanical mounting comprises a comfort structure. This serves, as the name suggests, to improve patient comfort despite the substantially incompressible frame structure. The comfort structure is arranged over, that is above, the frame structure on the patient side. Therein, the supporting structure is still configured higher than the frame structure and the comfort structure together. If the supporting structure is compressed to the height of the comfort structure, the patient lies substantially on the comfort structure.

In further embodiments, it is provided for this purpose that the supporting structure has a lower hardness than the comfort structure. However, the comfort structure is preferably also configured to be compressible, and is preferably also formed from a foam material. The comfort structure shields the frame structure from the patient. Preferably, the comfort structure is formed from a foam material that is 20% to 30% harder than the foam material of the supporting structure. The comfort structure serves in some embodiments, apart from the substantially incompressibly formed frame structure and the compressible carrier structure, to absorb the force applied in total on the measuring system and thereby to position the patient pleasantly softly. The base area of the comfort structure corresponds to the base area of the frame structure. The comfort structure thus covers the frame structure, preferably completely, on its side facing away from the carrier structure.

In some example embodiments, the frame structure, or the frame structure and the comfort structure together, have a recess facing in the direction of the sensor electrode. The recess is preferably formed on the side facing away from the carrier structure by the frame structure and/or the frame structure and the comfort structure. The recess becomes larger upwardly, that is, toward the patient. For example, the recess can have a triangular form in cross-section or can be configured as a circular sector comprising 90°. The recess advantageously serves to accommodate parts of the supporting structure when it is compressed. In other words, the supporting structure can give way into the recess, depending upon the compression. In this way, an overlap between the sensor electrode and/or the supporting structure and the frame structure and/or the comfort structure is prevented, so that an input of the excessive force portion into the comfort/frame/carrier structure is ensured.

In at least one example embodiment, the supporting structure is formed from a foam material which, for small compression changes, in particular height changes in the millimeter range, creates a substantially constant counterforce. In this way, it can be ensured that even with slight patient movement, as occurs during the measurement of bioelectrical signals and/or caused by breathing or heartbeat, in an ideal case, no capacitance change occurs at the sensor electrode (or relatively low change), since by this means the equilibrium between the force components which causes a compression of the supporting structure to the height of the frame structure and/or the comfort structure and the counterforce applied by the supporting structure remains largely unchanged. Preferably, a viscoelastic foam material is utilized herein. It has a high level of energy absorption. Viscoelastic foam materials generate a larger counterforce during a compression than after the compression. In a compression region of approximately 40%, the counterforce advantageously changes only very slightly. In preferred embodiments, the viscoelastic foam material can be configured as GV50/30 viscofoam.

In some example embodiments, the material of the supporting structure is selected taking account of the height ratio between the frame/comfort structure and the supporting structure so that a compression to the height of the frame structure and/or the comfort structure is achieved by a weight force of approximately 10 N to 50 N, preferably approximately 30 N acting upon the sensor electrode and/or the supporting structure. This applies both in embodiments of the supporting structure made of a foam material or a compressible plastics material.

In some embodiments, in particular embodiments that are provided for an ECG mat, the height of the supporting structure corresponds to approximately 1.5 to 2 times the height of the frame structure or the frame structure together with the comfort structure. It is also ensured thereby that through the compression of the supporting structure to the height of the frame structure and/or the comfort structure, the supporting structure is brought into a compression region of approximately 40% in which the counterforce developed changes only unnoticeably during patient movement.

It is also achieved thereby that the counterforce developed by way of the supporting structure remains substantially constant during the signal acquisition despite small patient movement and/or a compression caused thereby of the patient tissue lying thereon or thereagainst by a few millimeters and therefore the measurement signal remains undisturbed.

In some embodiments, the frame structure can have a height of between 2 cm and 6 cm. The supporting structure can have a height of between 4 cm and 10 cm.

In other embodiments, in particular in embodiments of the measuring system which is provided for an ECG chest belt, the supporting structure can preferably have a height of 5 mm to 10 mm, whereas the frame structure can have a height of 3 mm to 9 mm. It has been found empirically that a height difference between the supporting structure and the frame structure of 1 mm to 2 mm is sufficient for ECG measurements by a chest belt in order, nevertheless to acquire stable measurement signals since on the ribcage, in particular close to the ribs of a patient, there is only little (uneven and/or compressible) tissue, which could compress the supporting structure further below the height of the frame structure and thereby influence the contact pressure on the sensor electrode. The heights given enable, overall, an advantageously low structural height of the measuring system, which is comparable with the height of typical chest belt ECG electronics and is readily wearable under clothing.

In an alternative embodiment, the measuring system comprises a compression film that is arranged such that it precompresses the supporting structure by approximately 30% to 40%. The compression film preferably extends beneath the sensor electrode and above the supporting structure and is preferably constructed layered.

The compression film can be made, for example, of different materials, preferably a plastics material. In particular, it can comprise one of the following materials: polyvinyl chloride (PVC), polyethylene (PE) and polypropylene (PP). In preferred embodiments, the compression film has a thickness of 50 μm to 500 μm, corresponding to the thickness of a typical adhesive film. This ensures the durability of the film without impairing the function of the measuring system.

In some embodiments, the compression film is connected to the side of the frame structure facing toward the patient and/or to the comfort structure and/or is fastened there. The compression film covers over at least the supporting structure completely or has substantially the same base area as the frame structure and the supporting structure together. In other embodiments, the compression film can also stretch over a plurality of adjacent measuring systems. The compression film advantageously causes the supporting structure to be always in a precompressed state, in particular, regardless of a current orientation or position of a patient. Particularly preferably, the pretension is set so that the supporting structure is compressed by 40%. In this region, a variance of the counterforce built up by the supporting structure for small compression changes is only slight. In addition, the use of a compression film saves space since the height of the measuring system becomes reduced. In some embodiments, the height difference between the supporting structure and the frame structure/comfort structure can thus be reduced to 0.5 cm to 1 cm.

At least another example embodiment relates to a differential voltage measuring system for measuring bioelectrical measurement signals from a patient. The voltage measuring system has at least two signal measuring circuits, each in accordance with a useful signal path and each comprising a sensor electrode. At least one of the signal measuring circuits, preferably all the signal measuring circuits included, comprise a measuring system for movement compensation, as described above.

The differential voltage measuring system acquires bioelectrical signals, as mentioned in the introduction, for example, from a human or animal patient. For this purpose, it has a plurality of measuring lines and/or useful signal paths. These connect, for example, sensor electrodes as individual cables which are attached to the patient for acquisition of the signals, to further components of the voltage measuring system, that is, in particular an electronics system that serves to evaluate and/or display the acquired bioelectrical signals, in particular heartbeat signals.

Differential voltage measuring systems are known to a person skilled in the art according to their basic function, so that a detailed explanation is omitted here. In particular, they can be configured as an electrocardiogram (ECG), electroencephalogram (EEG) or electromyogram (EMG) device. Particularly preferably, the differential voltage measuring system is integrated into a capacitive ECG mat comprising a plurality of sensor electrodes on which the patient simply lies for a measurement signal acquisition. In further embodiments, the differential voltage measuring system is integrated into an ECG chest belt.

The differential voltage measuring system according to example embodiments has at least one signal measuring circuit according to example embodiments which comprises a measuring system according to example embodiments. The differential voltage measuring system according to example embodiments therefore also shares the advantages of the signal measuring circuit according to example embodiments.

In an example embodiment of the differential voltage measuring system, a plurality, that is at least two, signal measuring circuits are included which have a measuring system according to example embodiments. The carrier structures, intermediate structures, comfort structures, frame structures and/or the compression film of the at least two measuring systems are each configured integrally with one another.

In particular, if the differential voltage measuring system is configured mat-like and several, that is significantly more than two, sensor electrodes are arranged two-dimensionally beside one another, carrier structures, intermediate structures, comfort structures, frame structures and/or the compression film of the respective measuring systems can each be configured in a coherent way. This minimizes the assembly effort and reduces the component complexity.

In a configuration of a chest belt, the differential voltage measuring system can have exactly two sensor electrodes and corresponding signal measuring circuits, which are both integrated into the chest belt.

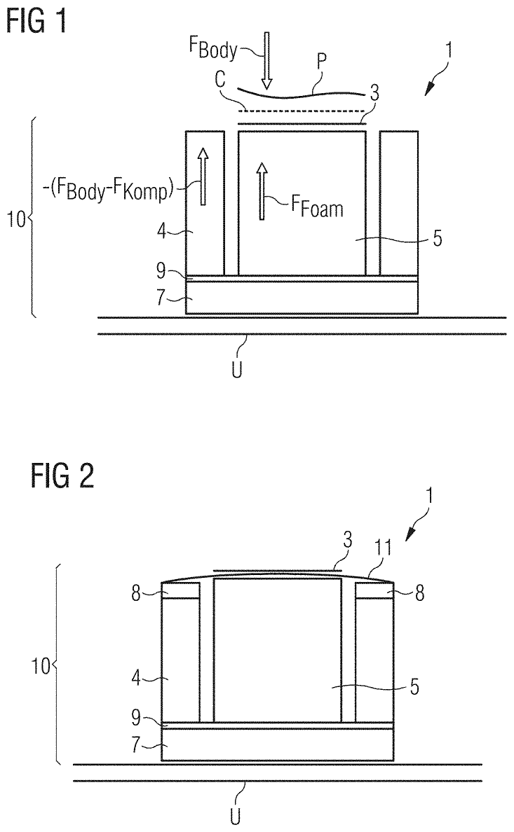

FIG. 1 shows a view of a measuring system 1 in an exemplary embodiment in a first loading state. The measuring system 1 shown serves to measure bioelectrical signals of a patient P, who, in this embodiment, is positioned on the measuring system 1. Thus a weight force FBody acts upon the measuring system 1. Said measuring system comprises a sensor electrode in the form of a flat electrode 3. This is covered, for example, by a textile layer C of the clothing of the patient P. Furthermore, a mechanical mounting 10 is included for the sensor electrode 3. This is configured to be compressible at least partially, that is, at least in some regions, i.e. it can be compressed. The mechanical mounting 10 comprises a frame structure 4 and a supporting structure 5. The supporting structure 5 is arranged beneath the sensor electrode and has a base area corresponding at least to the base area of the sensor electrode 3, so that the sensor electrode 3 lies completely on the supporting structure 5. The more accurately the two base areas are adapted to one another, the better. However, the supporting structure can also have a larger base area than the sensor electrode 3. The supporting structure carries the sensor electrode 3. The mechanical mounting 10 is fastened to a substrate U of the measuring system 1 and supports the sensor electrode 3 against the substrate U. The substrate is formed, for example, by the underside/the support surface of a captive patient mat for measuring ECG signals. The frame structure 4 at least partially surrounds the supporting structure 5. In this embodiment, the frame structure 4 is provided on two sides of the supporting structure 5. In alternative embodiments, the frame structure 5 can be a structure enclosing the supporting structure 5.

The supporting structure 5 is configured higher than the frame structure 4. Thus, in the unloaded state, it extends further from the substrate U in the direction of the patient P than the frame structure 4.

In this embodiment, the supporting structure 5 is designed to be compressible. Expressed more precisely, the supporting structure 5 is formed of a compressible foam material.

The frame structure 4 can also be formed from a compressible material, in particular a foam material (promotes patient comfort). It can however also consist of an incompressible material, for example, a plastics material or wood. In any event, the supporting structure 5 has a lower hardness than the frame structure 4. The supporting structure 5 consequently has a greater compressibility than the frame structure 4. The frame structure 4 must in any event be selected to be so hard that it can hold the supporting structure 5 constantly and in the relatively long term substantially at the height of the frame structure 4.

Through a force component FComp of the weight force FBody of the patient P, the supporting structure 5 is now compressed to substantially the height of the frame structure 4. The predominant/remaining portion of the weight force (FBody−FComp) is transmitted to the less compressible, or incompressible frame structure 4. The force component FComp acting upon the sensor electrode 3 and/or the supporting structure 5 causes a counterforce FFoam generated by the supporting structure 5 in the direction of the patient P, which corresponds in quantity to the force component FComp, thus, FComp=−FFoam. This counterforce FFoam now remains constant even during patient movement, for example, due to shifting weight, heartbeat and/or breathing, since a maximum compression of the supporting structure 5 is preset by the frame structure 4. A variation of the weight force FBody acting has an effect, on the frame structure 4, but no longer on the supporting structure 5.

In an ideal case, disturbances on the measurement signal are thereby eliminated. In practice, slight variations in FComp and FFoam remain, mainly caused by different tissue compressibilities in the patient P, which can be different from patient to patient and between different support positions of a patient P.

The measuring system 1 shown comprises as a further component of the mechanical mounting 10, a carrier structure 7. This is arranged beneath the supporting structure 5 and the frame structure 4 and above the substrate U. In this embodiment, the carrier structure 7 covers the substrate in a full-surface manner over the base area of the measuring system 1. In this embodiment, the carrier structure 7 is also formed from a compressible foam material and serves, firstly, despite the hard frame structure 4 to achieve a comfortable support for the patient P. For this purpose, the carrier structure 7 is also configured softer than the frame structure 4, but harder than the supporting structure 5. In the present case, the carrier structure 7 is configured 25% harder than the supporting structure 5. A portion of the weight force (FBody−FComp) acting upon the frame structure 4 can be conducted into the carrier structure 7, compress it and cause the frame structure 4 and the supporting structure 5 together with the sensor electrode 3 to give way together, dependent upon the weight force. Secondly, the carrier structure 7 permits, particularly in developments in which the measuring system 1 is integrated multiple times into a capacitive patient mat, an equalization of a different height level of the patient surface. For example, by the carrier structure 7, a lordosis form can be compensated for so that despite the variable height level, all the individual measuring systems 1 of the patient mat can provide a measurement signal. In lordosis, the carrier structure 7 would hardly be compressed, but with patents P having a straight back, correspondingly more. The interplay and/or the functionality of the frame structure 4 and the supporting structure 5 is also retained in this case. In the event of a patient movement also, the carrier structure 7 would be compressed to a differing extent. The counterforce FFoam=−FComp acting on the sensor electrode would nevertheless be kept substantially constant.

In order to achieve a particularly even force input over the base area of the measuring system 1 into the carrier structure 7, in this embodiment, the mechanical mounting 10 comprises an intermediate layer 9. This connects the carrier structure 7 to the frame structure 4 and the supporting structure 5. This means it is also formed in a full-surface manner above the carrier structure 7, at least over the base area of the measuring system 1. The intermediate layer 9 provides, in particular, for a distribution of movement-related, highly locally acting, force peaks onto further regions of the carrier structure, whereby an inclination or tilting of the supporting structure 5 and/or the sensor electrode 3 remains minimized and thereby the sensor surface always remains oriented substantially plane-parallel to the patient surface in order to ensure an optimum signal capture.

In some embodiments, in particular developments in which the measuring system 1 is integrated multiple times, for example, into a capacitive patient mat, the frame structure 4, the intermediate layer 9 and the carrier structure 7 of a number of measuring systems (arranged adjacently) can be configured integrally or connected to one another. For example, for the entire patient mat, a single carrier structure 7 and a single intermediate layer 9 lying thereupon can be provided, upon which the frame and supporting structures 4 and 5 of the plurality of measuring systems 1 are then arranged. To this extent, the carrier structure 7 and the intermediate layer 9 can have the same dimensions, specifically those of the base area of the measuring system as far as the base area of a capacitive patient mat.

Herein, the base area of the supporting structure is square with the dimensions 3 cm×3 cm in accordance with the base area of the sensor electrode 3. The height of the supporting structure 5 is 8 cm. In the present case, the supporting structure consists of a viscoelastic foam material with a high degree of energy absorption, in particular, the foam material GV 50/30. This is distinguished in that it generates a substantially constant counterforce for small compression changes.

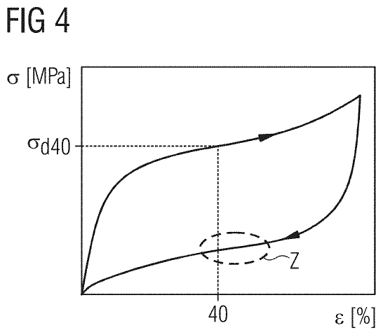

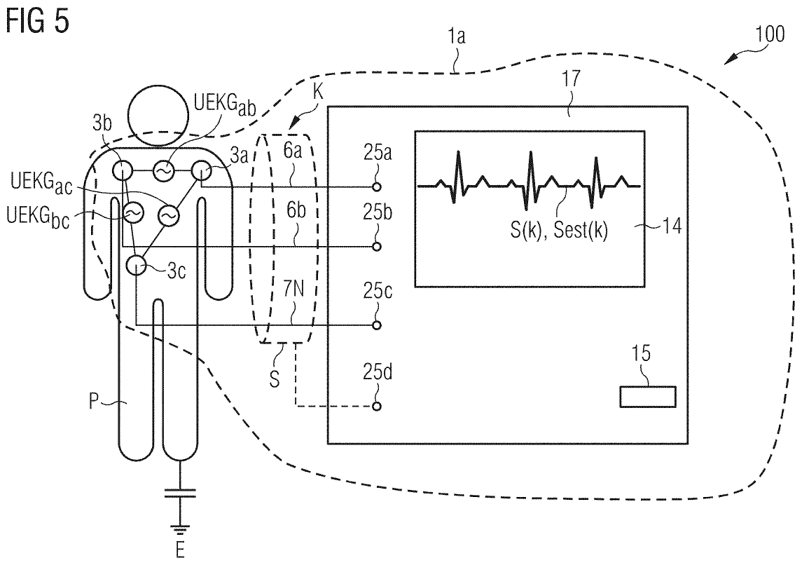

FIG. 4 shows by way of example, a compressive strength curve for a supporting structure material in the form of a viscoelastic foam material of a measuring system 1 in an exemplary embodiment. After a compression, the viscoelastic foam material generates a substantially smaller counterforce than during the compression. In a target region Z for the compression which lies in the region around 40%, this counterforce is approximately constant. In this target region, the supporting structure 5 must be brought by way of a patient weight-induced compression to the height of the frame structure 4. To this extent, the height of the supporting structure 5 should be approximately 1.4 to 1.6 times the height of the frame structure 4. In this respect, the frame structure 4 also has a height in the embodiment according to FIG. 1 of 4.5 cm.

In general, the selection of the supporting structure foam material and/or the heights of the supporting structure 5 and the frame structure 4 is made so that 10 N to 50 N of the weight force component FComp is sufficient to compress the supporting structure 5 of a measuring system 1 to the height of the frame structure 4. Even in light patients P, this causes a sufficient compression of the supporting structure 5 and brings about a quantitatively corresponding pressing force FFoam on the sensor electrode 3, which enables a stable signal acquisition.

Furthermore, the selection of the supporting structure foam material and/or the heights of the supporting structure 5 and the frame structure 4 are selected so that a height variance of the supporting structure 5 in the region of approximately 1 mm to 5 mm, typically caused by a weight force-related compression of patient tissue, substantially without a change of FFoam can be intercepted in order to prevent disturbing influences on the measurement signal.

The frame structure further has a depth of 3 cm corresponding to the sensor electrode 3 and/or the supporting structure 5 and a thickness of 1.5 cm. In some embodiments of a capacitive patient mat, at least the measuring systems 1 arranged directly adjoining the frame structures can be configured integrally or connected to one another. This advantageously increases the contact area for the patient and thereby increases patient comfort. The frame structure 4 consists here of a substantially incompressible plastics material. The carrier structure 7 has a height of 7 cm and consists of a viscoelastic foam material. The intermediate layer has a height of 0.5 cm and consists of a substantially incompressible material, in particular a fleece or a knitted fabric.

The individual structures/layers of the mechanical mounting 10 can be welded or glued to one another. Alternatively, at least the supporting structure 5 can be inserted, in each case, between the frame structures 4 or in a frame structure 4 and can thereby be fastened.

In further embodiments, the measuring system 1 is configured also to capture patient movements in the horizontal direction, that is substantially parallel to the sensor electrode area. For this purpose, the supporting structure 5 can be movably mounted in the frame structure 4, for example, at a separation from the frame structure 4 of up to 5 mm in each direction. In this way, the sensor electrode 3 can move with the body of the patient P. The supporting structure 5 is herein firmly connected at its underside to the intermediate layer 9, preferably heat welded. The supporting structure 5 is configured to carry out a shear movement during movement of the patient P in the horizontal direction. It can also be provided to mount a slippage-preventing support/layer on the frame structure 4 or the comfort structure 8 in order to minimize a movement of the clothing C of the patient P.

FIG. 2 shows a view of a measuring system 1 in a further exemplary embodiment. The measuring system 1 differs from the measuring system shown in FIG. 1 by a comfort structure 8 included in the mechanical mounting 10 and arranged above the frame structure 4. The comfort structure 8 serves, in particular, also for enhancing patient comfort. In this embodiment, the supporting structure 5 is higher than the frame structure 4 and the comfort structure 8 together. The comfort structure preferably covers the frame structure 4 completely. In other words, the base areas of the frame structure 4 and the comfort structure 8 correspond to one another. The comfort structure 8 is also formed here from a compressible material, preferably a foam material and thus forms a softer contact area facing toward the patient P. The comfort structure 8 also has a lower compressibility than the supporting structure 5. It is thereby ensured that in any event, the supporting structure 5 is firstly compressed to the height of the comfort and/or frame structure 8, 4, before a force input into the comfort and/or frame structure 8, 4 takes place. The comfort structure 8 is also configured to be 25% harder herein than the supporting structure 5. The comfort structure has a height herein of 2 cm.

In alternative embodiments, the comfort structure 8 can also be significantly thinner, for example, 0.5 cm to 1 cm and then can be configured less compressible. For example, the comfort layer in these embodiments consists of a fleece-like material and forms only a softer covering of the frame structure.

No weight force lies on the measuring system 1 shown herein. However, the embodiment of the measuring system 1 shown herein comprises a compression film 11 that is arranged such that it precompresses the supporting structure 5 by approximately 30% to 40%. The compression film 11 therefore causes the supporting structure 5 to be situated, even without loading by the patient weight, in the compression target region Z in which a compression change brings about only a small change in the counterforce FFoam. In this way, the structure and/or the height of the measuring system can advantageously be reduced without having to dispense with the advantages of example embodiments.

The compression film 11 consists in this embodiment of a polyvinyl chloride layer with a thickness of 50 μm. In this embodiment, it is fastened onto the outer sides of the comfort structure 8 and is dimensioned so that it brings about the desired precompression of the supporting structure 5. In other embodiments, in particular when many measuring systems are integrated into a capacitive patient mat, a compression film can be provided for a plurality of, in particular all, the measuring systems, which effectively causes a precompression of the supporting structures 5 for all the covered-over measuring systems 1.

In this embodiment, the sensor electrode 3 is arranged and fastened on the compression film 3, in particular heat-welded.

In general, it is the case that the more constant the counterforce FFoam (corresponding to the compression target region Z) applied by the supporting structure despite the varying weight force of the patient P acting upon the measuring system, the smaller the height difference between the supporting structure 5 and the frame structure 4 and possibly the comfort structure 8 can be selected to be. A height difference set by the compression film 11 of 0.5 cm to 1 cm between the supporting structure 5 and the frame structure 4 is then entirely sufficient to be able to exploit the advantages of the invention.

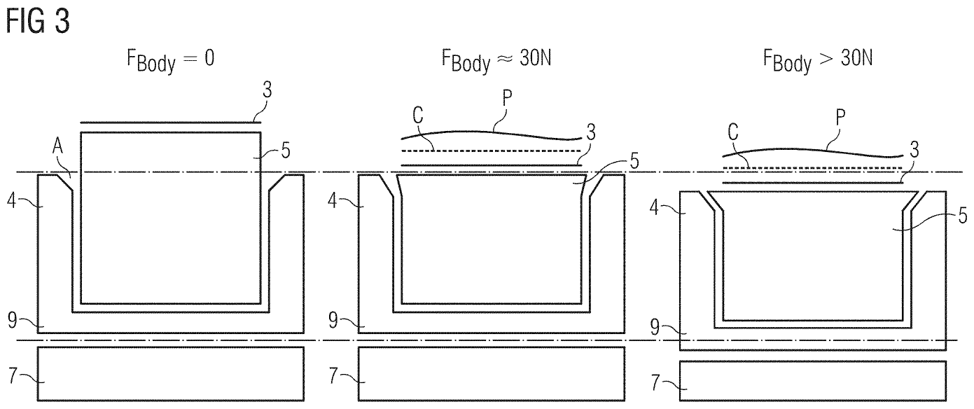

FIG. 3 shows a view of a measuring system in a further exemplary embodiment relating to different loading states corresponding to applied weight forces of FBody=0,

### Fbody≈

### 30 N and FBody>30 N.

A peculiarity of this exemplary embodiment is that the intermediate structure 9 is configured integrally with the frame structure 5. Both together form a basket-like shape into which the supporting structure 5 is received, in particular, fastened by a plug-in connection. A gluing and/or welding can herein advantageously be dispensed with. In this exemplary embodiment the intermediate layer 9 is preferably configured from the same relatively or totally incompressible material as the frame structure 4. Both structures thus provide for an even force input into the compressible carrier structure 7 arranged thereunder.

Furthermore, the measuring system 1 shown here comprises a frame structure 4 with a recess A facing in the direction of the sensor electrode 3 into which the supporting structure 5 can give way, depending upon the compression. This has the advantage that the contact area formed by the supporting structure 5 for the sensor electrode 3 can advantageously be held planar and parallel to the carrier structure 7 and/or the substrate U.

FBody=0: No weight force acts on the measuring system 1. The supporting structure 5 extends, together with the sensor electrode 3 far out of the frame structure 4. The recess A of the frame structure 4 is empty.

Fbody≈30 N: A patient P lies with clothing C on the measuring system 1. A weight force of approximately 30 N acts on the measuring system 1. The supporting structure 5 is compressed to the height of the frame structure 4 (upper dashed line). The supporting structure develops a counterforce FFoam acting upon the sensor electrode which remains substantially constant for as long as the supporting structure 5 maintains the height of the frame structure 4. The supporting structure 5 expands slightly into the recess A. The sensor electrode 3 therefore continues to lie flat on the supporting structure 5.

FBody>30 N: The patient moves, changes his position or breathes, or suchlike. There acts (at least partially) a weight force of greater than 30 N. The remaining weight force (FBody −FComp) acts via the incompressible frame and intermediate structure 4, 9 and causes a compression of the carrier structure 7. The frame, supporting and intermediate structure 4, 5, 9 are sunk below the original height level (see upper and lower dashed lines). However, the height of the supporting structure 5 does not change further due to the incompressibility of the frame structure 4. FFoam also remains largely constant in this loading state. The supporting structure 5 now fills the recess A completely. The sensor electrode 3 therefore continues to lie flat on the supporting structure 5.

FIG. 5 shows a view of a differential voltage measuring system 100 arranged on a patient P in the form of an ECG system. The general functioning of an ECG system is described below on the basis of FIG. 5. The voltage measuring system 100 comprises an ECG device 17 with its electrical terminals and sensor electrodes 3a, 3b, 3c connected thereto via cables K in order to measure ECG signals S(k) on the patient P. At least one, preferably all the sensor electrodes 3a, 3b, 3c can be configured as part of a measuring system 1 as described in relation to the following figures.

In order to measure ECG signals S(k), at least one first electrode 3a and one second electrode 3b are needed which are mounted at, on or under the patient P. By way of signal measuring cables K, the electrodes 3a, 3b, 3c are connected via terminals 25a, 25b, usually plug-in connectors, to the ECG device 17. The first electrode 3a and the second electrode 3b together with the signal measuring cables K therein form a part of a signal acquisition unit, with which the ECG signals S(k) can be acquired.

A third electrode 3c serves as a reference electrode in order to create a potential equalization between the patient P and the ECG device 17. Classically, this third electrode 3c is attached to the right leg of the patient (“right leg drive” or RLD). However, it can also, as here, be positioned at a different site. Furthermore, using further terminals (not shown), a plurality of further contacts can be mounted on the ECG device 17 for further terminal leads (potential measurements) on the patient P and used for the formation of suitable signals.

The voltage potentials UEKGab, UEKGbc and UEKGac form between the individual electrodes 3a, 3b, 3c and are used for measuring the ECG signals S(k).

The directly measured ECG signals S(k) are displayed on a user interface 14 of the ECG device 27.

During the ECG measurement, the patient P is coupled at least capacitively to the ground potential E (represented by a coupling to the right leg).

The signal measuring cables K which lead from the first sensor electrode 3a and the second sensor electrode 3b to the ECG device 17, are a portion of the useful signal paths 6a, 6b and the corresponding sensor lines. The signal measuring cable K which leads from the electrode 3c to the ECG device 17 herein corresponds to a portion of a third useful signal path 7N. The third useful signal path 7N can serve, in particular, to transfer disturbance signals that were coupled in via the patient P and the electrodes.

The cables K have a screening S which is represented here schematically as a dashed cylinder surrounding all the useful signal paths 6a, 6b, 7N. The screening does not however have to surround all the cables K together, rather the cables K can also be separately screened. However, the terminals 25a, 25b, 25c preferably each have a pole integrated for the screening S. These poles are then brought together to a common screening terminal 25d. The screening S is configured therein, for example, as a metal foil surrounding the conductor of the respective cable K, which however, is insulated from the conductor.

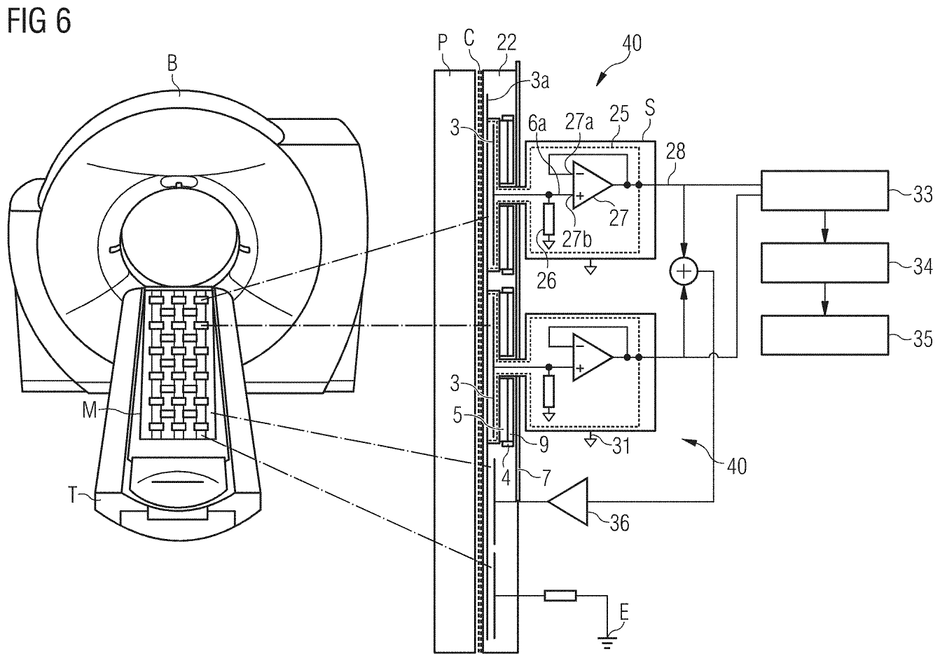

In addition, the ECG device 17 can have an external interface 15 in order to provide, for example, a terminal for a printer, a storage facility and/or even a network. The ECG device 17 has signal measuring circuits 40 associated with each of the terminals 25a, 25b (see e.g. FIG. 6) according to exemplary embodiments. The signal measuring circuits 40 are each themselves connected via a ground switch 31 to ground E.

FIG. 6 shows a view of a differential voltage measuring system 100 comprising two signal measuring circuits 40 in an exemplary embodiment. The two signal measuring circuits have an identical structure, so that for the sake of clarity, corresponding components have largely been provided with reference signs only once.

The arrangement of a single sensor 3 and/or a single sensor electrode 3 of the signal measuring circuit 40 is illustrated here in the form of a capacitive ECG measuring circuit. The patient P and the sensor electrode 3 are situated in spatial proximity to one another. Stated more precisely, patient P lies here on a patient table T of an imaging modality B in the form of a computed tomography system. A capacitive patient mat M, on which the patient P is positioned, is arranged on the patient table. The patient mat M comprises a plurality of signal measuring circuits 40. The mat M can alternatively be configured as an electron pad which can be arranged, in particular, in a backrest of an examination or treatment chair. Two of the signal measuring circuits 40 are described in greater detail below. Patient P can be provided, for example, with a textile cover C. Optionally lying thereupon is a cover 22 which is transparent to X-rays. The sensor electrode 3 is not in direct electrical contact with the patient P, but is electrically insulated from the patient P at least by a sensor covering 3a. However, a capacitive coupling of an ECG signal is not impaired by the sensor cover 3a. The sensor electrode 3, a sensor line 6a extending from the sensor electrode 3 to an operational amplifier 27, and the measuring circuit 40 comprising the operational amplifier 27 are surrounded by a so-called active protective screen 25 and preferably a screening S. The operational amplifier 27 is configured as a so-called follower. I.e. the negative input 27a of the operational amplifier 27 is coupled to the output 28 of the operational amplifier 27. In this way, a high virtual input impedance is achieved for the operational amplifier 27 at the positive input 27b. This means that due to the voltage adaptation between the output 28 and the positive input 27b, a current flows between the sensor 3 and the active protective screen 25. Furthermore, the positive input 27b of the operational amplifier 27 is maintained, with the aid of a resistor 26 connected to the measuring device ground (also called “measuring ground”), at an electrical bias voltage. Thereby, the positive input can be set to a desired measurement potential. In this way, DC components are suppressed. This is desirable since the sensor electrode 3 should couple, above all, capacitively and a potential approaching it should be prevented.

The signal measuring circuits 40 shown each comprise a measuring system 1, for example, according to other drawings, each comprising a mechanical mounting 10 with a supporting structure 5, a frame structure 4, a carrier structure 7 and an intermediate structure 9. The active guard 25 and the shield S further each enclose the sensor electrode 3 in order to screen it effectively. The active guard 25 and the shield S further surround the sensor line 6a and, together with it interpenetrate the support, intermediate and carrier structures 5, 9, 7 on the route to the operational amplifier 27. The carrier structure 7 is herein configured integrally and effectively acts for both signal measuring circuits 40. Alternative arrangements of the sensor line 6a are naturally also conceivable.

A further electrode is also provided in the patient mat M shown here for at least capacitive coupling of the patient to the ground potential E.

A further electrode and/or the associated measuring circuit 36 functions in the patient mat M as a reference electrode and/or as a so-called driven neutral electrode (DNE).

The differential voltage measuring system 100 further comprises a switching apparatus in the form of a switch matrix 33. In the presence of a large number of sensor electrodes 3, it serves to select which of the sensor electrodes is used for a further signal processing.

The differential voltage measuring system 100 further comprises a signal processing apparatus in the form of a signal processing box 34. This is configured to carry out a pre-processing of the acquired measurement signals in order to remove disturbance components. The signal processing apparatus 34 can be configured to carry out a standard processing with frequency-based filters such as band-pass or band-stop filters, but also an extended disturbance suppression such as, for example, in the German patent application DE 102019203627A.

Furthermore, the differential voltage measuring system 100 comprises a trigger apparatus 35. This is configured to carry out a method for recognizing a heartbeat of the patient P and/or the heart rhythm in order therefrom to generate control signals comprising an item of trigger and/or start time point information for a medical imaging system. On the basis of the control signals of the trigger apparatus 35, the imaging apparatus calculates the time points for an image acquisition.

The embodiments in FIGS. 1 to 5 relate, in principle, to arrangements in which a patient P is in a lying position and his weight force FBody acts upon at least one measuring system 1. However, example embodiments are not restricted thereto. A measuring system 1 is also advantageously usable for sitting positions of the patient P or suchlike. In particular, as a component of a variable backrest, the contact pressure also varies very greatly dependent upon the posture/position of the patient P and this can also be compensated for according to at least some example embodiments as described above.

The construction of a measuring system is more complex than the conventional use of a foam material as an underlay for a sensor electrode 3. The construction is, however, economical overall to realize and can be designed to be X-ray transparent with suitable materials.

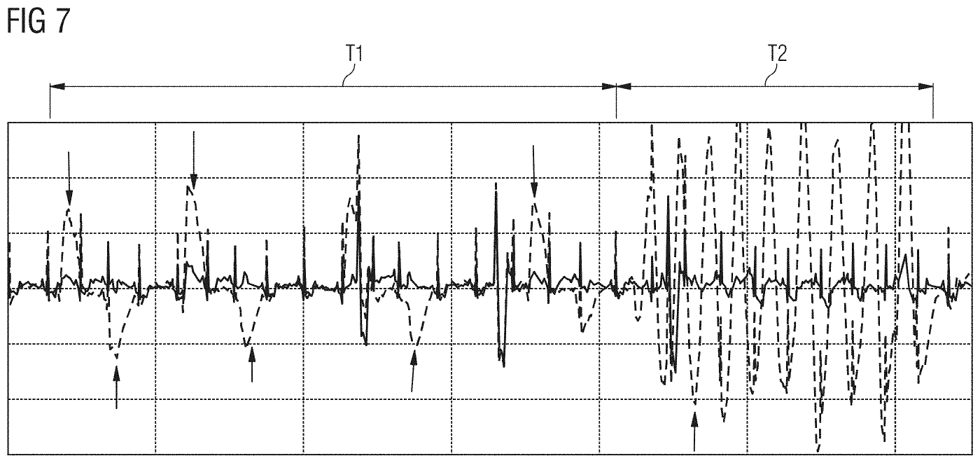

FIG. 7 shows a comparison of ECG signals acquired with a conventionally mounted capacitive ECG sensor (dashed line) and with a capacitive ECG sensor with a mechanical mounting according to example embodiments (solid line). In one experiment, it was possible to make clear the influence of the mechanical mounting according to example embodiments. In the experiment, the conventional ECG sensor was mounted on the left shoulder blade of a subject and the ECG sensor configured according to example embodiments was mounted parallel to it on the right shoulder blade of the subject.

In a first time interval T1, the subject took four deep breaths, each causing a body movement. They manifested in the dashed curve as disturbing influences arising as very marked amplitude deflections. The solid curve has only reduced signal deviations at the same points (see arrows).

In a second time interval T2, the subject carried out, by way of a test, a plurality of weight shifts from the right side of the body to the left. Here also, the reduced sensitivity of the ECG sensor compared with the conventional sensor is clearly evident. The movement-related disturbing influences are apparent in the solid-line curve only as signal oscillations that are greatly reduced in comparison with the dashed curve.

At least one example embodiment thereby permits a demonstrably improved level of capacitive ECG signal quality during patient movement.

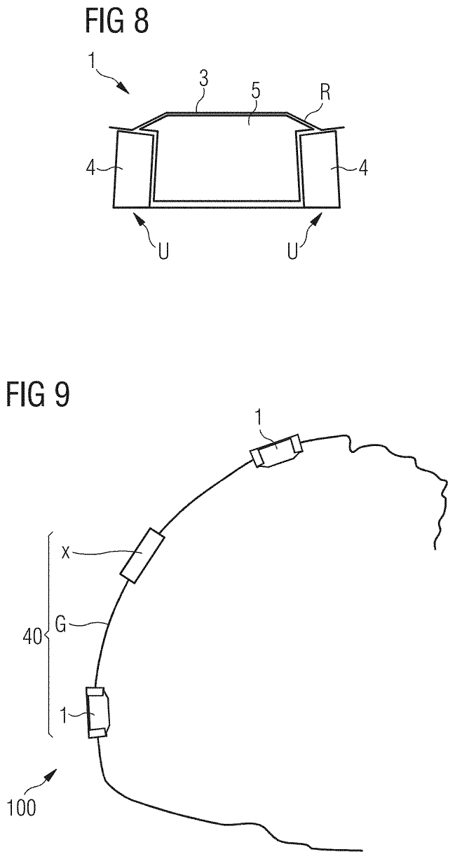

FIG. 8 shows a view of a measuring system 1 in another exemplary embodiment. This measuring system 1 is adapted to the ambient conditions of a capacitive ECG chest belt G (FIG. 9).

FIG. 9 shows correspondingly a view of a differential voltage measuring system 100 comprising two signal measuring circuits 40 in the form of an ECG chest belt G. The two measuring systems 1 of the chest belt G are herein connected in this case to a shared electronics system X and each form a signal measuring circuit 40 therewith. The chest belt G can be configured open, as shown, or closed. It is placed around the chest and/or the upper body of a patient P, wherein the sensor electrodes 3 therein rest against the upper surface of the patient P. The belt itself can consist of a textile fabric, a woven fabric, a knitted fabric or fleece, leather or rubber.

The measuring system 1 comprises a conductive sensor electrode 3 which is arranged facing toward the patient (not shown here). It has an area from 1 cm×1 cm up to 2 cm×5 cm. The sensor electrode is configured as a flat electrode with a layered construction. The measuring system 1 comprises a supporting structure 5 on which the sensor electrode 3 is positioned and fastened. The base areas of the sensor electrode 3 and the supporting structure 5 largely correspond, wherein that of the supporting structure 5 can also be constructed somewhat larger. The supporting structure 5 is herein constructed compressible, specifically with a force-compensating foam material, for example, a polyurethane (PUR) foam material and/or a plastics material. The measuring system 1 also comprises a frame structure 4 which surrounds the supporting structure 5 on at least two sides. In contrast to the supporting structure 5, the frame structure 4 is constructed from a hard, i.e. incompressible, material, for example, a polyethylene (PE) plastics material. The supporting structure 5 has a higher degree of hardness than the frame structure 4 and extends, together with the sensor electrode 3, over the height of the frame structure. When the sensor electrode 3 is contacted, a pressure/pressing force is applied to the sensor electrode 3 and/or the supporting structure 5. The supporting structure foam material is compressed by the force component FComp to the height of the frame structure 4. A force acting beyond this force component is primarily conducted into the frame structure 4. In this embodiment, the supporting structure 5 and the frame structure 4 form the mechanical mounting 10 which, supports the sensor electrode 3 against a substrate U of the measuring system 1.

The mechanical mounting 10 is advantageously suitable for use in signal measuring circuits that are integrated into an ECG chest belt since, located over the ribs of a patient P, there is only a little (compressible) tissue that could press the supporting structure 5 further into the frame structure 4 as far as the height thereof. This advantageously enables a smaller structural height overall of 0.5 cm to 1 cm, which corresponds to the height of typical chest belt ECG electronics systems.

The height difference between the supporting structure 5 and the frame structure 4 in this embodiment can itself be restricted to 1 mm to 2 mm for the load-free state due to the evenness of the ribcage and the small electrode area itself. protrude beyond the supporting structure.

In the embodiment shown here, the sensor electrode 3 is bound to a slip-proof film R, for example, made of rubber. The slip-proof film primarily covers the frame construction 4. Therefore, on loading by a patient body, it is pressed particularly strongly against said body.

The sensor electrode 3 consists of a conductive material, preferably with a surface resistance of greater than 100 kOhm. In this embodiment, the sensor electrode 3 must not be configured slip-proof itself or be coated since it does not absorb the main pressing force. It therefore has a, in particular, more durable, smooth, metallic surface with a constant conductivity.

The frame construction 4 can consist in some embodiments of a conductive material. Apart from the holding function of the frame, this simultaneously causes a screening of the sensor electrode 3 against electric fields. For this purpose, the conductive material of the frame structure 4 is either bound to an electrical line at the potential of an associated signal measuring circuit or to an electrical layer at the potential of the patient body.

The slip-proof layer R does not have to fulfil any electrical requirements. Example embodiments therefore offer a large degree of freedom in relation to the materials used and permits an improved optimization of the slip-proofing as compared with conventional solutions in which the largest pressing force acted upon the sensor electrode 3 and this had to be configured electrically conductive and slip-proof. Therefore, there results an improved protection against slippage of the chest belt G. The embodiment of the frame structure 4 as an electrical screening additionally protects, in particular, against severe electrostatic charges as are commonly caused by polyester-containing sports clothing.

The measuring system 1 shown herein can also greatly reduce disturbances to an ECG signal caused by pressure changes, as are caused by breathing or pressure on the chest belt G by clothing or accessories.

Where it has not yet explicitly been set out, although useful, individual exemplary embodiments, individual sub-aspects or features thereof can be combined or exchanged with one another without departing from the scope of example embodiments. Advantages of some example embodiments also apply without explicit mention, where transferable, to other exemplary embodiments.

1. A measuring system for measuring bioelectrical signals of a patient, the measuring system comprising:
a sensor electrode; and
a mechanical mounting for the sensor electrode, the mechanical mounting being at least partially compressible and comprising a frame structure and a supporting structure,
wherein
the mechanical mounting is fastened to a substrate of the measuring system and supports the sensor electrode against the substrate,
the supporting structure is arranged beneath the sensor electrode,
the frame structure at least partially surrounds the supporting structure, and
the supporting structure is configured higher than the frame structure.

2. The measuring system as claimed in claim 1, wherein the mechanical mounting is formed at least partially of foam material.

3. The measuring system as claimed in claim 1, wherein the supporting structure has a lower hardness than the frame structure.

4. The measuring system as claimed in claim 1, wherein the mechanical mounting comprises a carrier structure which extends beneath the supporting structure and the frame structure.

5. The measuring system as claimed in claim 4, wherein the mechanical mounting comprises an intermediate structure which connects the carrier structure to the frame structure and the supporting structure.

6. The measuring system as claimed in claim 5, wherein the intermediate structure is configured integrally with the frame structure.

7. The measuring system as claimed in claim 5, wherein the mechanical mounting comprises a comfort structure arranged above the frame structure, wherein the supporting structure is higher than the frame structure and the comfort structure together.

8. The measuring system as claimed in claim 7, wherein the supporting structure has a lower hardness than at least one of the comfort structure or the carrier structure.

9. The measuring system as claimed in claim 7, wherein the frame structure, or the frame structure and the comfort structure, have a recess facing in the direction of the sensor electrode, in which the supporting structure can give way depending upon a compression.

10. The measuring system as claimed in claim 1, wherein the supporting structure is formed from a foam material, the foam material configured to create a substantially constant counterforce based on a compression change.

11. The measuring system as claimed in claim 7, wherein the height of the supporting structure is approximately 1.4 to 1.6 times the height of the frame structure or the frame structure and the comfort structure together.

12. The measuring system as claimed in claim 1, comprising a compression film, the compression film being arrange to precompress the supporting structure by approximately 30% to 40%.

13. A signal measuring circuit for a differential voltage measuring system for measuring bioelectrical signals of a patient, the signal measuring circuit comprising:
the measuring system as claimed in claim 1;
a measuring amplifier circuit; and
a sensor line between the measuring amplifier circuit and the sensor electrode.

14. A differential voltage measuring system for measuring bioelectrical signals of a patient, which has at least two signal measuring circuits, each signal measuring circuit corresponding to one useful signal path, wherein at least one of the signal measuring circuits comprises the measuring system as claimed in claim 1.

15. A differential voltage measuring system, comprising:
at least two signal measuring circuits, each of the measuring circuits including,
a sensor electrode, and
a mechanical mounting for the sensor electrode, the mechanical mounting being at least partially compressible and comprising a frame structure and a supporting structure,
the mechanical mounting is fastened to a substrate of the measuring system and supports the sensor electrode against the substrate,
the supporting structure is arranged beneath the sensor electrode,
the frame structure at least partially surrounds the supporting structure, and
the supporting structure is configured higher than the frame structure; and
the frame structures of the at least two measuring systems are each configured integrally with one another.

16. The measuring system as claimed in claim 2, wherein the supporting structure has a lower hardness than the frame structure.

17. The measuring system as claimed in claim 2, wherein the supporting structure is formed from a foam material, the foam material configured to create a substantially constant counterforce based on a compression change.

18. The measuring system as claimed in claim 8, wherein the supporting structure is formed from a foam material, the foam material configured to create a substantially constant counterforce based on a compression change.

19. The measuring system as claimed in claim 9, wherein the supporting structure is formed from a foam material, the foam material configured to create a substantially constant counterforce based on a compression change.
